(19) United States
(12) Patent Application Publication
(10) Pub. No.: US 2025/0115838 A1
(43) Pub. Date: Apr. 10, 2025

(54) SAMPLING APPARATUS FOR CELL CULTURES

(71) Applicant: SABEU GMBH & CO. KG, NORTHEIM (DE)

(72) Inventor: DENNIS BENKMANN, KALTENKIRCHEN (DE)

(73) Assignee: SABEU GMBH & CO. KG, NORTHEIM (DE)

(21) Appl. No.: 18/716,958

(22) PCT Filed: Nov. 15, 2022
(86) PCT No.: PCT/EP2022/081991

§ 371 Date: Dec. 16, 2024

(30) Foreign Application Priority Data
Dec. 9, 2021 (DE) 10 2021 132 560.9

(51) Int. Cl. C12M 1/32; C12M 1/00; C12M 3/00

(52) U.S. Cl. CPC C12M 23/12; C12M 23/38; C12M 23/46

(57) ABSTRACT

A sample device includes a multiwell plate having single wells which are firmly connected to the multiwell plate. Each single well has a sample room for a nutrient solution which is delimited from the other single wells, a wall which delimits the single well on an outside, a bottom which delimits the single well on a bottom which adjoins the wall, and an opening which is arranged opposite to the bottom and via which the single well is accessible. The wall has an opening contour which surrounds the opening and which delimits the wall, the opening contour having grooves each of which are arranged to have a respective supporting arm of a sample insert be inserted thereinto. The single wells are arranged so that the walls of neighboring single wells do not have grooves arranged directly opposite to each other.

### CROSS REFERENCE TO PRIOR APPLICATIONS

This application is a U.S. National Phase application under 35 U.S.C. § 371 of International Application No. PCT/EP2022/081991, filed on Nov. 15, 2022 and which claims benefit to German Patent Application No. 10 2021 132 560.9, filed on Dec. 9, 2021. The International Application was published in German on Jun. 15, 2023 as WO 2023/104449 A1 under PCT Article 21(2).

### FIELD

The present invention relates to a sample device for cell cultures with a multiwell plate which has a plurality of single wells firmly connected to the multiwell plate.

### BACKGROUND

A sample device of this type is described in DE 10 2012 100 583 B4. The single wells there have a sample room for a nutrient solution which is separated from the other single wells and into each of which a sample insert is suspended.

### SUMMARY

An aspect of the present invention is to provide a sample device of the type mentioned above which, due to its design, is less prone to errors in use.

In an embodiment, the present invention provides a sample device which includes a multiwell plate comprising a plurality of single wells which are firmly connected to the multiwell plate. Each single well of the plurality of single wells comprises a sample room for a nutrient solution which is delimited from the other plurality of single wells, a wall which is configured to delimit the single well on an outside, a bottom which is configured to delimit the single well on a bottom which adjoins the wall, and an opening which is arranged opposite to the bottom and via which the single well is accessible. The wall comprises an opening contour which surrounds the opening and which delimits the wall, the opening contour comprising grooves each of which are configured to have a respective supporting arm of a sample insert be inserted thereinto. The plurality of single wells are arranged so that the walls of neighboring single wells do not have grooves arranged directly opposite to each other.

### BRIEF DESCRIPTION OF THE DRAWINGS

The present invention is described in greater detail below on the basis of embodiments and of the drawings in which:

FIG. 1 shows a sample device according to the present invention with a multiwell plate with inserted sample inserts and with a lid;

FIG. 2 shows top view of the multiwell plate from FIG. 1;

FIG. 3 shows a sectional view of the object in FIG. 2 along line III-III;

FIG. 4 shows a detailed enlargement of the object in FIG. 2 from a perspective in the direction of arrow IV in FIG. 2;

FIG. 5 shows the lid from FIG. 1 showing the inside of the lid; and

FIG. 6 shows a sectional view of the object in FIG. 5 along line VI-VI.

### DETAILED DESCRIPTION

The sample device according to the present invention comprises a multiwell plate which has a plurality of single wells firmly connected to the multiwell plate. Each single well has a sample room for a nutrient solution which is separated from the other single wells and is bordered on the outside by a wall and below by a bottom adjoining the wall. The sample room is accessible via an opening opposite the bottom, which is surrounded by an opening contour bordering the wall. Each opening contour of the wall of a single well has associated grooves for inserting supporting arms of a sample insert. The grooves provide that a sample insert inserted into a single well in a certain position cannot subsequently slip into a new position that deviates therefrom. The supporting arms inserted in the grooves and the sample insert above them are prevented from subsequently changing position via the grooves. The immutability of the position of the sample insert is of great importance, for example, in TEER measurements on cell cultures. The embodiment according to the present invention also provides that contaminants not located on the supporting arms of a sample insert are additionally introduced into a neighboring single well when the sample insert is inserted into a single well. The embodiment according to the present invention also prevents a sample insert with a supporting arm from mistakenly being inserted into a groove of a single well that is not assigned to that single well but to a neighboring single well. The sample insert would otherwise be mounted at an angle and measurements would be falsified.

In an advantageous embodiment of the present invention, each of the walls can, for example, have a raised safety margin compared to the perimeter area immediately adjacent to the respective single well. The perimeter area is understood to be the material portion of the multiwell plate that is directly adjacent to the wall of the single well. This can be a flat labeling area next to the single wells or a bottom plate of the multiwell plate. This embodiment provides that sample material or contaminants that accidentally end up next to a single well do not penetrate into the sample room of the single well. This also provides that supporting arms of a sample insert positioned in the grooves of the single wells do not fall below a safety distance from the perimeter area. This also prevents an accidental contamination on the perimeter area from reaching the supporting arms. The safety margin advantageously has an inner side which faces the sample room and an outer side opposite thereto, with the outer side extending further in the direction of a bottom plane in which the bottoms of the single wells are positioned than the perimeter area of the multiwell plate directly adjacent to the outer side.

In an embodiment of the present invention, the safety margin of each single well can, for example, be raised by at least 0.5 mm, in particular by at least 1 mm, compared to the perimeter area immediately adjacent to the respective single well. The safety margin is drawn higher outside the grooves than in the area of the grooves. The aforementioned minimum height of the safety margin is not, however, undercut even in the area of the grooves. The aforementioned advantages of the safety margin in counteracting contamination are thus maintained even if the height of the safety margin is reduced in the area of the grooves.

In an advantageous embodiment of the present invention, the sample device can, for example, comprise a lid which is adapted to the shape of the multiwell plate and the sample inserts with a hollow cylindrical housing having an upper housing opening on one side, a membrane-like housing bottom opposite the housing opening, and supporting arms arranged on the housing which project outwards and which are spaced apart from one another. The lid has holding-down devices on the inside which are positioned above each supporting arm when the sample device is closed and the lid is placed on the multiwell plate. The holding-down devices can, for example, be designed as lugs on the inside of the lid. The holding-down devices prevent the sample inserts from floating on a nutrient liquid in the sample room. The sample insert is pressed down into the nutrient liquid by the holding-down devices. This provides sufficient contact between the bottom of the sample insert and the nutrient liquid and prevents the supporting arms from leaving the grooves as a result of floating and thus bypassing the function of the grooves. The gas exchange between the sample room and the ambient air outside the sample device is also improved.

The supporting arms advantageously have at least one lug on the surface facing away from the housing bottom with the holding-down devices being positioned above these lugs. The lugs can, for example, have a spherical, rounded shape. The contact surface between the holding-down device and the lug on the supporting arm is thus minimized.

The distance between the holding-down devices and the supporting arms is advantageously no more than 0.3 mm when the sample device is closed and the lid is placed on the multiwell plate. Unwanted floating can thus be reduced to a minimum.

In an embodiment of the present invention, the holding-down devices can, for example, rest on the supporting arms in the closed state of the sample device with the lid placed on the multiwell plate. The undesired floating of the sample insert on the nutrient medium can thus be completely prevented.

In an advantageous embodiment of the present invention, each of the grooves in the opening contour has an insertion area in the upper region, which has two opposite limbs which are conical to each other and each enclose an angle of 15° to 45°, in particular of 25° to 35°, to a perpendicular to the bottom plane in which the bottoms of the single wells lie. The groove tapers downwards in the insertion area,. This tapering makes it easier to insert the sample insert into the multiwell plate. The possibility of incorrect insertion is reduced. The sample insert also occupies a narrowly defined end position as a result of the taper. The insertion area can, for example, extend over at least half the height of the groove.

The opening contour in the lower area of each groove advantageously has a fastening area in which the opening contour runs perpendicular to the bottom plane on both sides. The vertical course of the grooves in the fastening area makes it more difficult for the supporting arms to leave the grooves unintentionally. At the same time, however, the advantages of the tapering of the grooves in the upper insertion area are retained. The opposing opening contours of the walls of the grooves run parallel to each other in the fastening area.

In an embodiment of the present invention, the fastening area can, for example, extend over a height of 0.1 mm to 1 mm, in particular from 0.2 mm to 0.4 mm. The height of the fixation area in the aforementioned ranges is sufficient to fulfill the aforementioned function of the fastening area and, at the same time, the upper insertion area also remains sufficiently dimensioned to fulfill its aforementioned function.

In an embodiment of the present invention, the sample device can, for example, comprise sample inserts with a hollow cylindrical housing having an upper housing opening on one side, a membrane-like housing bottom opposite the housing opening, and supporting arms arranged on the housing and projecting outwards and spaced apart from one another, the width of the supporting arms being 0.1 mm to 0.5 mm, in particular 0.25 mm to 0.35 mm, less than the width of the deepening at the level at which the supporting arms are inserted in the grooves. The supporting arms therefore have very little lateral play in the grooves so that the positioning selected by inserting the sample insert into the sample room of the single well is hardly changed by external influences. Measurements in which the positional invariability of the cell material in the sample insert relative to the multiwell plate is important, such as TEER measurements, are therefore less prone to error.

The grooves advantageously have a depth of 1.5 mm to 3 mm, in particular 1.7 mm to 2.3 mm, for example, from 1.9 mm to 2.1 mm. This range of depth has proven to be particularly advantageous for achieving the aforementioned advantages of the grooves.

The multiwell plate can, for example, have 6, 12 or 24 single wells which are positioned next to each other in even rows.

Further details and advantages of the present invention are set forth in the embodiment example as shown in the drawings.

The features of the sample device shown below can also be the subject of the present invention in other combinations. Equally effective elements are provided with a uniform reference number in the following, where this is appropriate.

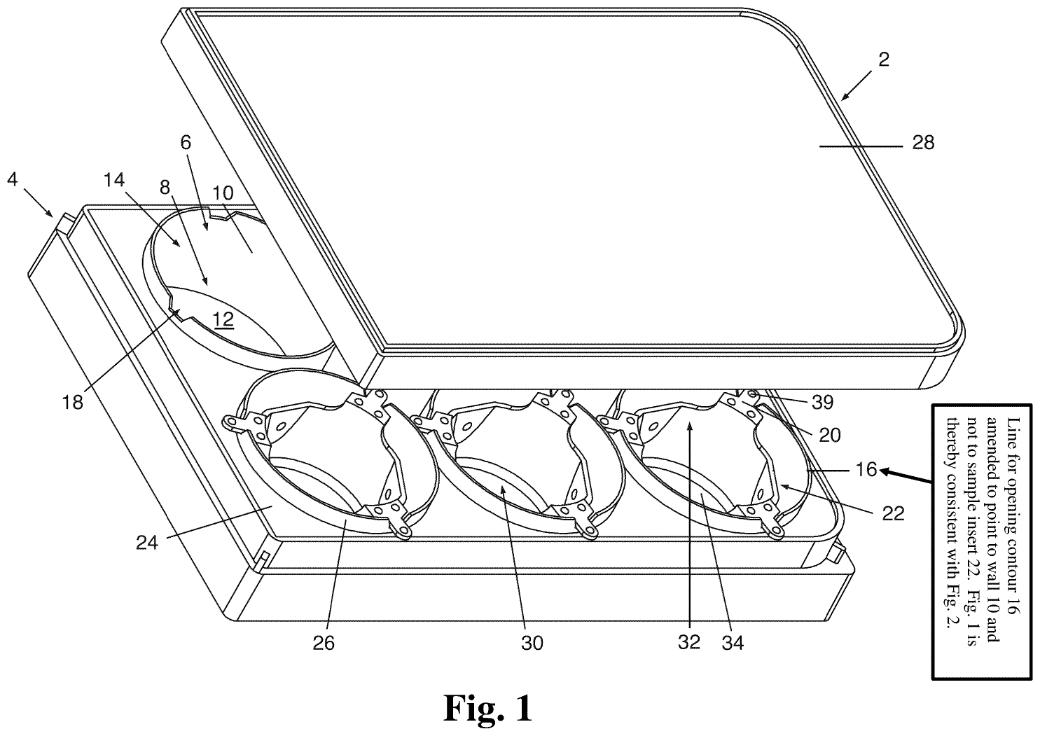

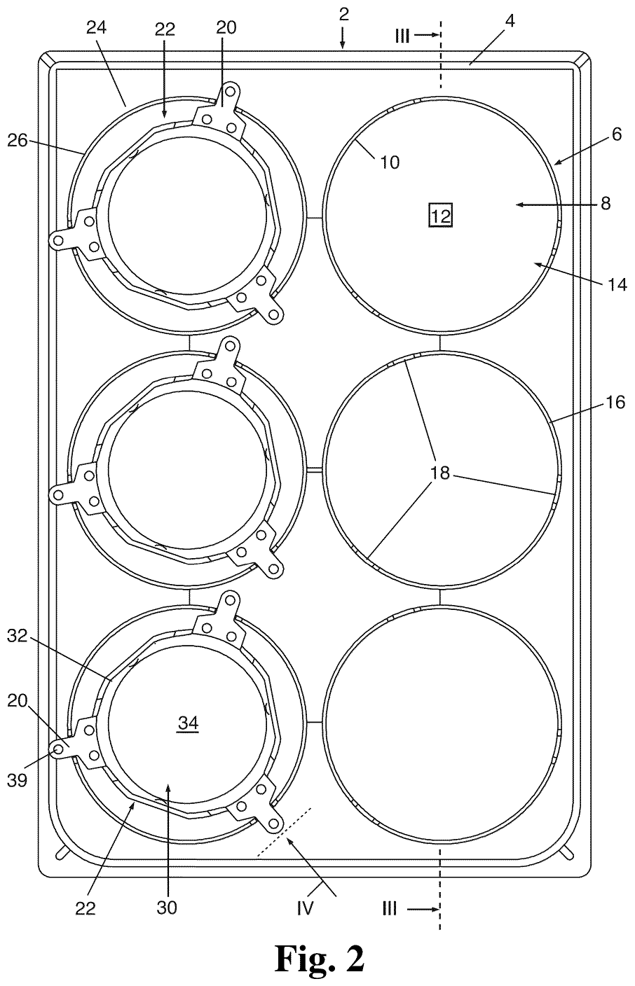

FIG. 1 shows a sample device 2 according to the present invention with a multiwell plate 4, which has a plurality of single wells 6 firmly connected to the multiwell plate 4. The multiwell plate 4 is shown in FIG. 2 in a plan view. Each single well 6 has a sample room 8 for a nutrient solution which is delimited from the other single wells 6, is bounded on the outside by a wall 10 and at the bottom by a bottom 12 adjoining the wall 10 and is accessible via an opening 14 opposite the bottom 12. The opening 14 is surrounded by an opening contour 16 delimiting the wall 10. Each opening contour 16 of the wall 10 of a single well 6 has grooves 18 associated with it for inserting supporting arms 20 of a sample insert 22 and is free of grooves 18 associated with adjacent single wells 6. In FIG. 1 and FIG. 2, three of the total of six single wells 6 are occupied by sample inserts 22.

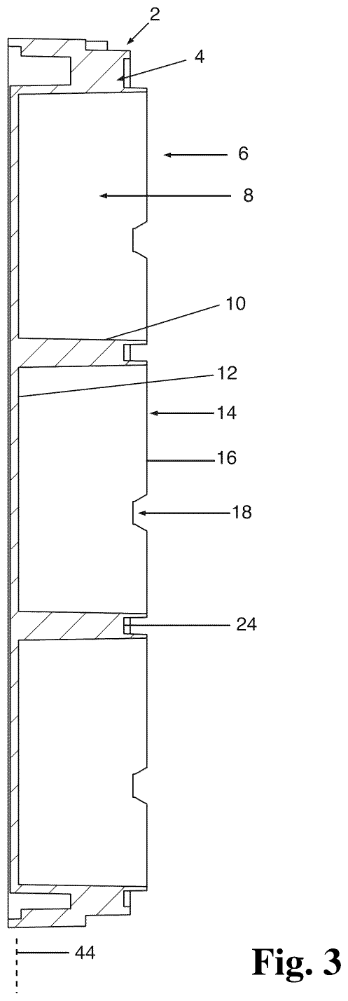

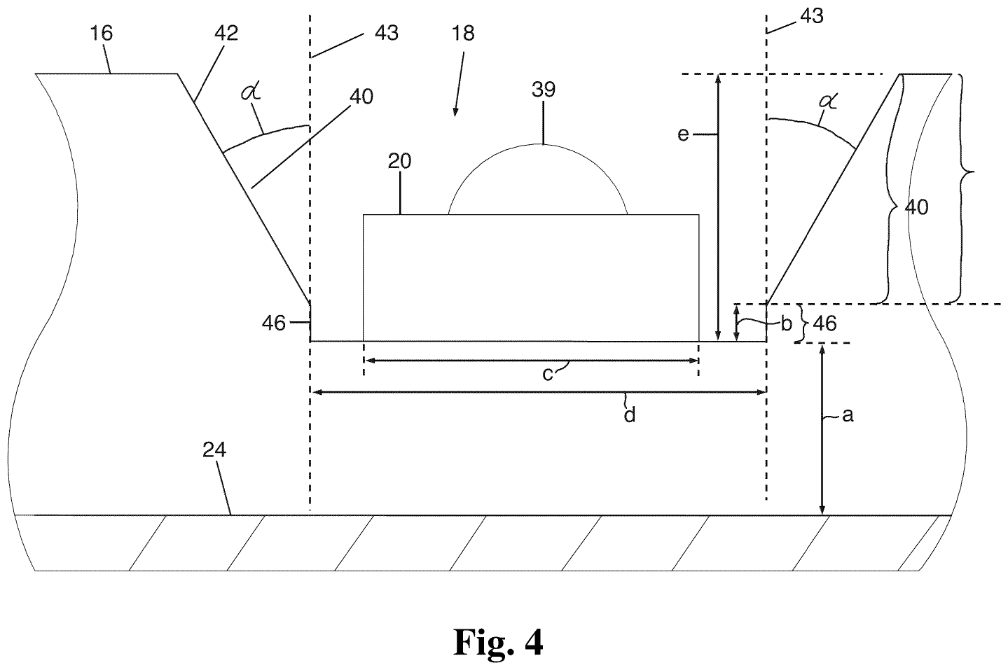

Each of the walls 10 has a raised safety margin 26 compared to the perimeter area 24 immediately adjacent to the respective single well 6. The safety margin 26 can clearly be seen in the sectional view of the multiwell plate 4 in FIG. 3 and even better in the partially sectioned detailed view in FIG. 4. The safety margin 26 of each single well 6 is raised by at least 0.5 mm, in particular by at least 1 mm, compared to the perimeter area 24 immediately adjacent to the respective single well 6. The perimeter area 24 is shown in section in FIG. 4. The aforementioned minimum height a of the safety margin 26 of at least 0.5 mm and in particular of at least 1 mm is there illustrated. This value is also not exceeded in the area of the grooves 18.

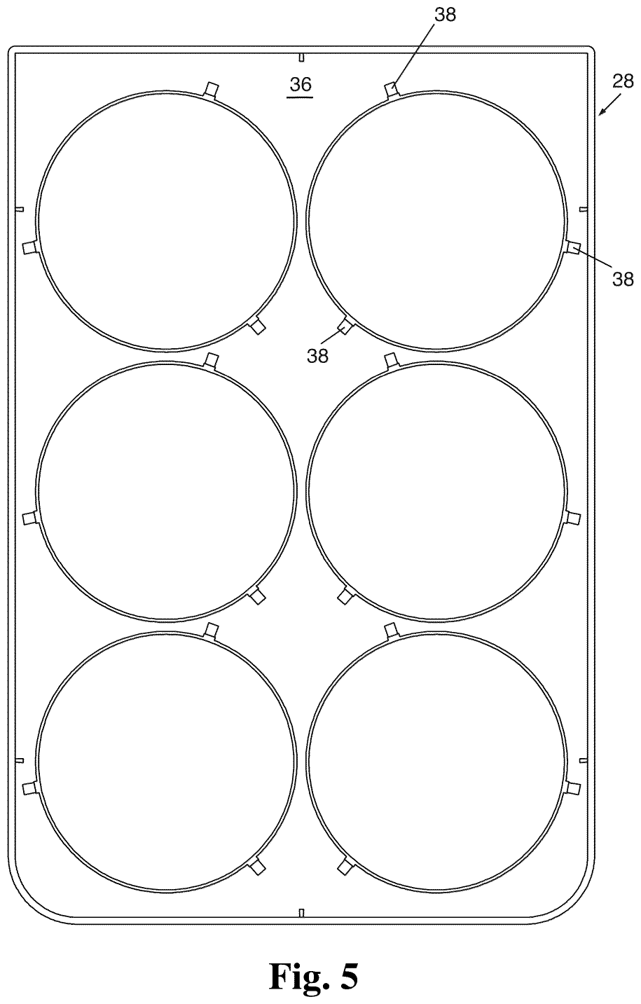

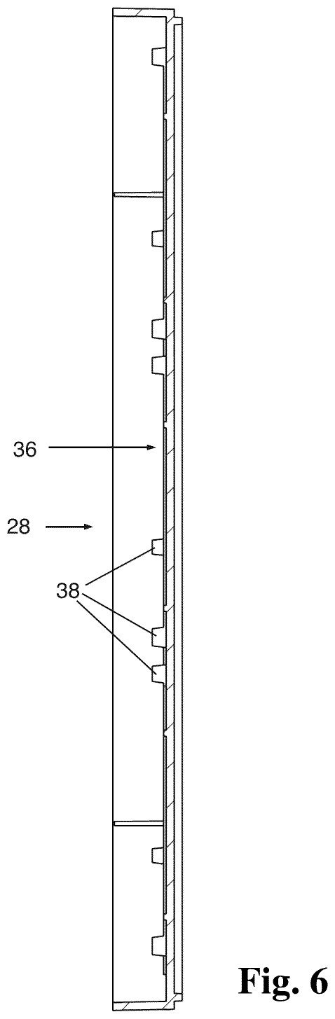

The sample device 2 in FIG. 1 also comprises a lid 28 which is adapted to the shape of the multiwell plate 4, and sample inserts 22 having a hollow cylindrical housing 32 having an upper housing opening 30 on one side, a membrane-like housing bottom 34 opposite the housing opening 30, and supporting arms 20 arranged on the housing 32 and projecting outwards and spaced apart from one another. The lid 28 is shown in FIG. 5 in a bottom view of the inside 36 of the lid 28. FIG. 6 shows a sectional view through the lid 28 according to line VI-VI. The lid 28 has holding-down devices 38 on the inside, each of which is positioned above a supporting arm 20 when the sample device 2 is closed with the lid 28 placed on the multiwell plate 4. The supporting arms 20 can, for example, have at least one lug 39 on the surface facing away from the housing bottom 34, the holding-down device 38 being positioned above this lug 39 in each case when the lid 28 is placed on the multiwell plate 4.

The distance between the holding-down devices 38 and the supporting arms 20 is not more than 0.3 mm in the closed state of the sample device 2 with the lid 28 placed on the multiwell plate 4. The holding-down devices 38 can, for example, rest on the supporting arms 20 in this state.

FIG. 4 shows a detailed view of the area of a groove 18. Each of the grooves 18 in the opening contour 16 has an insertion area 40 in the upper region, which has two opposite limbs 42 that extend conically to one another. The limbs 42 each include an angle α with a perpendicular 43 to the bottom plane 44, in which the bottoms 12 of the single wells 6 lie, of 15° to 45°, in particular of 25° to 35°.

The opening contour 16 has a fastening area 46 in the lower region of each of the grooves 18 in which the opening contour 16 extends perpendicular to the bottom plane 44 on both sides. The fastening area 46 extends over a height b of 0.1 mm to 1 mm, in particular from 0.2 mm to 0.4 mm.

FIG. 4 also shows a supporting arm 20 inserted into the grooves 18. The width c of the supporting arms 20 is 0.1 mm to 0.5 mm, in particular 0.25 mm to 0.35 mm, less than the width d of the groove 18 at the level at which the supporting arms 20 are inserted in the grooves 18.

The grooves 18 have a depth e of 1.5 mm to 3 mm, in particular 1.7 mm to 2.3 mm, as illustrated in FIG. 4.

The present invention is not limited to embodiments described herein; reference should be had to the appended claims.

### LIST OF REFERENCE CHARACTERS

2 Sample device
4 Multiwell plate
6 Single well
8 Sample room
10 Wall
12 Bottom
14 Opening
16 Opening contour
18 Groove
20 Supporting arm
22 Sample insert
24 Perimeter area
26 Safety margin
28 Lid
30 Housing opening
32 Housing
34 Housing bottom
36 Inside (of the lid)
38 Holding-down device
39 Lug (on supporting arm)
40 Insertion area
42 Limb
43 Perpendicular to the bottom plane
44 Bottom plane
46 Fastening area
α Angle between the limb and the perpendicular to the bottom plane
a Minimum height of the safety margin
b Height of the fastening area
C Width of the supporting arms
d Width of the groove
e Depth of the groove

What is claimed is:

1-11. (canceled)

12. A sample device comprising:
a multiwell plate comprising a plurality of single wells which are firmly connected to the multiwell plate, each single well of the plurality of single wells comprising,
a sample room for a nutrient solution which is delimited from the other plurality of single wells,
a wall which is configured to delimit the single well on an outside,
a bottom which is configured to delimit the single well on a bottom which adjoins the wall, and
an opening which is arranged opposite to the bottom and via which the single well is accessible,
wherein,
the wall comprises an opening contour which surrounds the opening and which delimits the wall, the opening contour comprising grooves each of which are configured to have a respective supporting arm of a sample insert be inserted thereinto, and
the plurality of single wells are arranged so that the walls of neighboring single wells do not have grooves arranged directly opposite to each other.

13. The sample device as recited in claim 12, wherein,
a perimeter area is arranged immediately adjacent to each of the plurality of single wells, and
the wall of each single well of the plurality of single wells further comprises a safety margin which has a height which is higher than the perimeter area.

14. The sample device as recited in claim 13, wherein the height of the safety margin of each single well of the plurality of single wells is 0.5 mm higher than the perimeter area immediately adjacent to each single well.

15. The sample device as recited in claim 12, further comprising:
a lid which is adapted to a shape of the multiwell plate, the lid comprising a plurality of holding-down devices each of which is arranged on an inside of the lid; and
a plurality of sample inserts, each sample insert of the plurality of sample inserts comprising a hollow cylindrical housing having an upper housing opening, a membrane-like housing bottom arranged opposite to the upper housing opening, and outwardly projecting, mutually spaced support arms arranged on the hollow cylindrical housing,
wherein,
a respective one of the plurality of holding-down devices of the lid is positioned above a respective one of the mutually spaced support supporting arms when the sample device is in a closed state with the lid placed on the multiwell plate.

16. The sample device as recited in claim 15, wherein a distance between the respective one of the plurality of holding-down devices of the lid positioned above the respective one of the mutually spaced support supporting arms, in the closed state of the sample device with the lid placed on the multiwell plate, is not more than 0.3 mm.

17. The sample device as recited in claim 16, wherein the respective one of the plurality of holding-down devices of the lid rests on the respective one of the mutually spaced support supporting arms in the closed state of the sample device with the lid placed on the multiwell plate.

18. The sample device as recited in claim 12, wherein,
the bottom of each single well of the plurality of single wells has a bottom plane, and
each of the grooves in the opening contour comprises an insertion area in an upper region, the insertion area comprises two limbs which are arranged opposite to each other and which extend conically with respect to one another, each of the two limbs having an angle of 15° to 45° to a perpendicular to the bottom plane.

19. The sample device as recited in claim 18, wherein the opening contour further comprises a fastening area in a lower region of each of the grooves, the opening contour being arranged perpendicular to the bottom plane on both sides in the fastening area.

20. The sample device as recited in claim 19, wherein the fastening area extends over a height of from 0.1 mm to 1 mm.

21. The sample device as recited in claim 12, further comprising:
a plurality of sample inserts, each sample insert of the plurality of sample inserts comprising a hollow cylindrical housing having an upper housing opening, a membrane-like bottom arranged opposite to the upper housing opening, and supporting arms which are arranged on the hollow cylindrical housing,
wherein,
the supporting arms are arranged to project outwards and to be are spaced apart from one another, and
a width of the supporting arms is 0.1 mm to 0.5 mm less than a width of the grooves at a level where the supporting arms are inserted in the grooves.

22. The sample device as recited in claim 21, wherein the grooves have a depth of 1.5 mm to 3 mm.
